(19) United States
(12) Patent Application Publication
(10) Pub. No.: US 2024/0060952 A1
(43) Pub. Date: Feb. 22, 2024

(54) DETERMINING A PROPERTY OF A SOIL USING A MACHINE

(71) Applicant: Caterpillar Paving Products Inc., Brooklyn Park, MN (US)

(72) Inventors: Mark Daniel BEHN, Saint Michael, MN (US); Robert K. IVERSON, Maple Grove, MN (US); Nicholas A. OETKEN, Brooklyn Park, MN (US); Kishore Sundara SRINIVASAN, Chennai (IN)

(73) Assignee: Caterpillar Paving Products Inc., Brooklyn Park, MN (US)

(21) Appl. No.: 17/821,274

(22) Filed: Aug. 22, 2022

(51) Int. Cl. G01N 33/24; E02D 1/04

(52) U.S. Cl. CPC G01N 33/246; E02D 1/04

(57) ABSTRACT

A controller may obtain measurement information for measuring a property of a soil of a job site. The controller may measure the property of the soil based on obtaining the measurement information. The controller may compare the measured property of the soil and a target property of the soil. The controller may generate one or more instructions that cause one or more other machines to perform one or more actions with respect to the soil. The one or more instructions may be generated based on comparing the measured property of the soil and the target property. The controller may provide the one or more instructions to the one or more other machines to cause the one or more machines to perform the one or more actions to adjust.

### TECHNICAL FIELD

The present disclosure relates generally to measuring a property of a soil and, for example, to determining a property of a soil using a machine.

### BACKGROUND

During a construction process, a level of moisture of a soil of a job site may be measured. Subsequent to measuring the level of moisture, a density of the soil may be measured. When the density reaches an appropriate density, a structure may be developed on the soil.

Typically, measuring the level of moisture and measuring the density are performed manually using a measuring device. The manual process may be performed for only a small section of the job site at a time (e.g., 1% of the job site). The manual process of measuring the level of moisture and the density must be repeated numerous times in order to obtain measurements for the entire job site. Therefore, the manual process is a time-consuming process. Accordingly, the manual process consumes computing resources associated with obtaining measurements manually, storage resources associated with storing the measurements, and/or network resources, among other examples.

Additionally, the manual process may be subject to errors. Due to the manual process, the measured level of moisture may not be an appropriate level of moisture and/or the measured density may not be an appropriate density for developing structures on the soil. In some instances, a structure may be built on a soil with an inappropriate density. Accordingly, such a structure may become subject to a compaction issue. The compaction issue may affect an integrity of the structure. In some instances, the compaction issue may be corrected using one or more machines. Correcting the compaction issue is a time-consuming process. Additionally, correcting the compaction issue is a costly process. For example, repairing a soil base (after a structure has been built on the soil bases) is a time-consuming and costly process. The structure may include a road and/or a building, among other examples. Generally, the later the discovery of the compaction issue occurs, the more time consuming and costly the correction process becomes. The correction process becomes more time consuming and more costly because equipment may need to be transported back to a location of the compaction issue as well as new material (e.g., soil, aggregate, among other examples) that may need to be transported to the location.

Japanese Patent Application Publication No. JP6919862 (the '862 publication) discloses a transmission type ground property measuring device for measuring the properties of the ground. While the'862 publication discloses the ground property measuring device, the '862 publication does not address issues associated with inappropriate levels of moisture and/or inappropriate densities resulting in compaction issues.

The machine of the present disclosure solves one or more of the problems set forth above and/or other problems in the art.

### SUMMARY

A method performed by a controller of a machine includes obtaining measurement information for measuring a property of a soil of a job site, wherein the property of the soil includes one or more of a level of moisture of the soil or a density of the soil; measuring the property of the soil based on obtaining the measurement information; comparing the measured property of the soil and a target property of the soil, wherein the target property of the soil includes one or more of a target level of moisture of the soil or a target density of the soil; generating one or more instructions that cause one or more other machines to perform one or more actions with respect to the soil, wherein the one or more instructions are generated based on comparing the measured property of the soil and the target property; and providing the one or more instructions to the one or more other machines to cause the one or more other machines to perform the one or more actions.

A controller associated with a first machine includes one or more memories; and one or more processors configured to: obtain measurement information for measuring a property of a soil of a job site, wherein the property of the soil includes one or more of a level of moisture of the soil or a density of the soil; measure the property of the soil based on receiving the measurement information; compare the property of the soil and a target property of the soil, wherein the target property of the soil includes one or more of a target level of moisture of the soil or a target density of the soil; generate an instruction that causes a second machine to perform an action with respect to the soil to adjust the property of the soil with respect to the target property, wherein the instruction is generated based on comparing the property of the soil and the target property; and provide the instruction to the second machine to cause the second machine to perform the action to adjust the property of the soil.

A machine comprising: a plurality of ground engaging members configured to propel the machine at a job site; a measuring unit configured to measure a property of a soil of the job site; and a controller configured to: obtain measurement information for measuring the property of the soil; measure the property of the soil, using the measuring unit, based on receiving the measurement information; compare a value of the property of the soil and value thresholds associated with a target property of the soil; generate an instruction based on comparing the value and the value thresholds, wherein the instruction is a first instruction based on the value satisfying a first value threshold of the value thresholds, wherein the instruction is a second instruction based on the value not satisfying a second value threshold, of the value thresholds, that is less than the first value threshold, or wherein the instruction is a third instruction based on the value not satisfying the first value threshold and satisfying the second value threshold; and provide the instruction to one or more machines.

### BRIEF DESCRIPTION OF THE DRAWINGS

FIG. 1 is a diagram of an example implementation described herein.

FIG. 2 is a diagram of an example system described herein.

FIG. 3 is a flowchart of an example processes relating to determining a property of a soil.

### DETAILED DESCRIPTION

Implementations described herein are directed to using a machine to measure a property of a soil of a job site. For example, the machine may measure a level of moisture of the soil and/or a density of the soil. The machine may be an autonomous machine or a semiautonomous machine. Using the machine to measure the level of moisture or the level of density enables measurements to be obtained for sections of the job site. A size of each section of the job typically exceeds a size of a section of a job site for which the property of a soil is measured using a manual process. Additionally, using the machine to measure the property of the soil generates measurements that are more accurate and/or which are collected more rapidly than measurements of the property of the soil obtained using the manual process.

In some examples, the machine may generate one or more instructions that cause one or more other machines to perform one or more actions with respect to the soil. The one or more instructions may be generated based on comparing the property of the soil (measured by the machine) and a target property of the soil. The machine may provide the one or more instructions to the one or more machines to cause the one or more machines to perform the one or more actions. The one or more actions may cause the soil to achieve the target property.

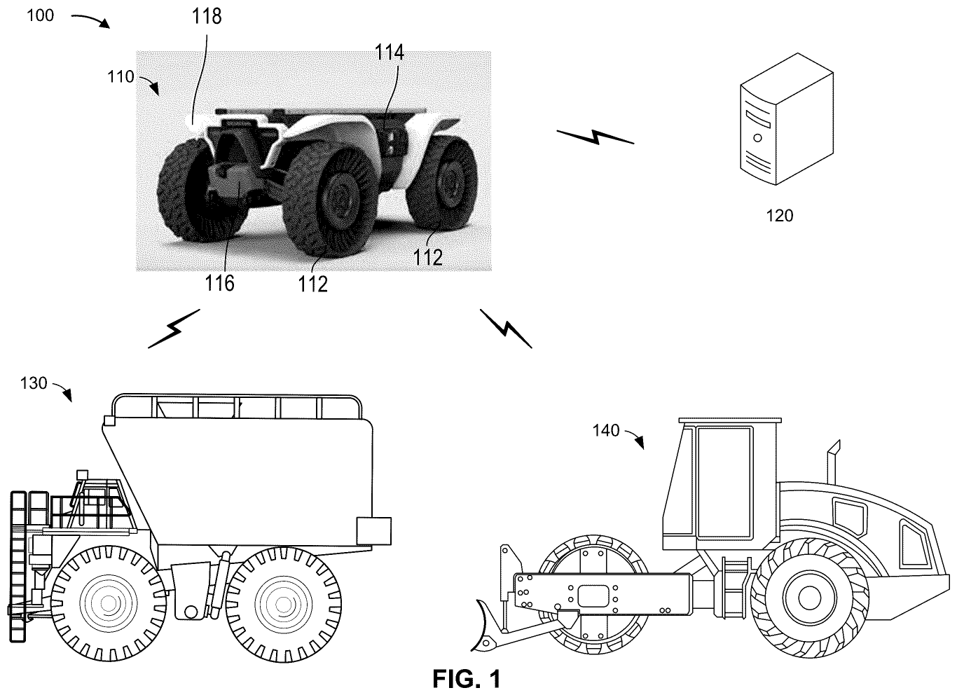

FIG. 1 is a diagram of an example implementation 100 described herein. As shown in FIG. 1, example implementation 100 includes a first machine 110, a back office system 120, a second machine 130, and a third machine 140. First machine 110 is illustrated as an unmanned ground vehicle configured to measure a property of a soil of a job site. Alternatively, first machine 110 may be another type of machine configured to measure the property of the soil. For example, first machine 110 may include an unmanned aerial vehicle.

As shown in FIG. 1, first machine 110 may include ground engaging members 112, a controller 114, a measuring unit 116, and a global positioning system (GPS) device 118. Ground engaging members 112 may be configured to propel first machine 110. Ground engaging members 112 may include wheels, tracks, rollers, and/or similar components, for propelling first machine 110.

Controller 114 (e.g., an electronic control module (ECM)) may control and/or monitor operations of first machine 110. For example, controller 114 may control and/or monitor the operations of first machine 110 based on signals from measuring unit 116, from GPS device 118, and/or from back office system 120, as described in more detail below. For instance, controller 114 may process instructions (e.g., from back office system 120) to measure the property of the soil and may cause soil property information, regarding the soil, to be provided to back office system 120, to second machine 130, and/or to third machine 140, among other examples. In some situations, the soil property information may include information identifying the property of the soil and/or information identifying a location of the soil.

Measuring unit 116 may include one or more devices configured to measure the property of the soil of the job site. Measuring unit 116 may include one or more devices configured to measure levels of moisture of soils, such as a moisture measurement meter, a radioactive device, and/or a probe penetration device, among other examples. Additionally, or alternatively, measuring unit 116 may include one or more devices configured to measure density of soils, such as a nuclear density gauge, a falling lightweight deflectometer, a ground penetrating radar, and/or plate load test equipment, among other examples. The ground penetrating radar may be used to measure density and soil mechanical properties, such as modulus of elasticity.

GPS device 118 may include one or more devices that are capable of receiving, generating, storing, processing, and/or providing signals that may be used to determine a location of first machine 110, a velocity of first machine 110, and/or a movement of first machine 110, among other examples. As an example, GPS device 118 may generate location data that may be used to determine the location of the soil being tested by first machine 110, or the position of first machine 110 as it moves about the job site. In some implementations, a motor grader or a dozer (or another machine) may define a work area and may provide a signal to first machine 110 to start work in the work area. The work area may be an area that has just been graded and is ready for compaction or moisture measurement. A compactor and first machine 110 may communicate so that first machine 110 knows a path of the compactor (e.g, a path traveled by the compactor). By knowing the path of the compactor, first machine 110 may measure soil after each pass of the compactor or after every other pass of the compactor.

Back office system 120 may include one or more devices that are capable of receiving, generating, storing, processing, and/or providing signals that may be used to monitor and/or control operations of machines located at the job site. In some examples, back office system 120 may provide instructions to first machine 110 to cause first machine 110 to measure the property of the soil. Additionally, back office system 120 may provide instructions to second machine 130 and/or third machine 140 to cause second machine 130 and/or third machine 140 to perform actions with respect to the soil based on the property of the soil measured by first machine 110.

As shown in FIG. 1, second machine 130 is illustrated as a water truck and third machine 140 is illustrated as a compactor. Alternatively, second machine 130 and third machine 140 may be other types of machines, such as a motor grader, a wheel loader, a mining truck, among other examples. In some situations, second machine 130 and/or third machine 140 may be configured to receive instructions that cause second machine 130 and/or third machine 140 to perform an action with respect to the soil. The action may adjust the property of the soil.

For example, second machine 130 and/or third machine 140 may be configured to provide water to the soil to increase the level of moisture of the soil, may be configured to break up the soil to decrease the level of moisture of the soil, and/or may be configured to compact the soil (e.g., in preparation for a paving operation), among other examples. Second machine 130 and/or third machine 140 may receive the instructions from first machine 110 and/or from back office system 120.

As indicated above, FIG. 1 is provided as an example. Other examples may differ from what was described in connection with FIG. 1.

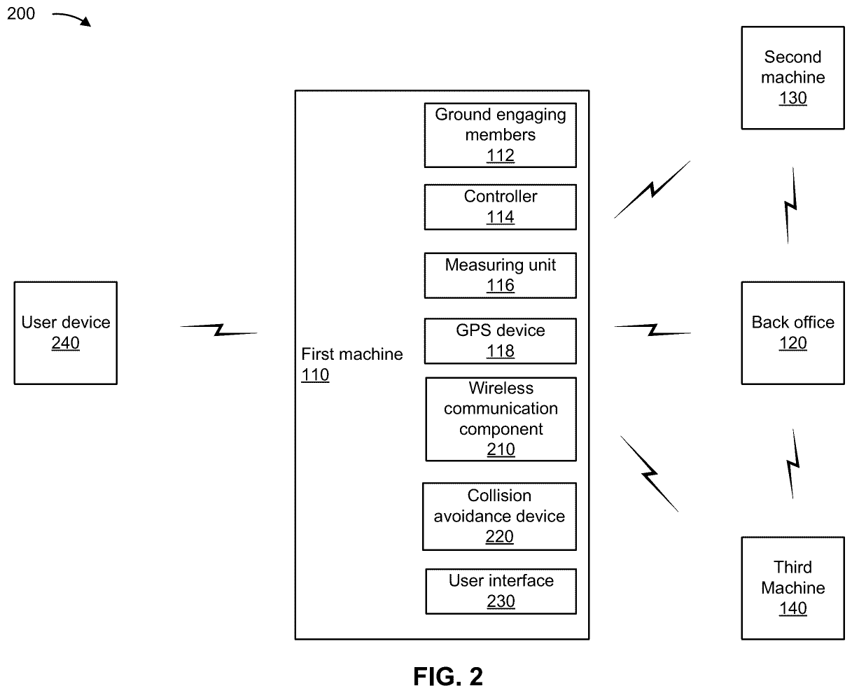

FIG. 2 is a diagram of an example system 200 described herein. As shown in FIG. 2, system 200 includes first machine 110, back office system 120, second machine 130, third machine 140, and a user device 240. Some elements of FIG. 2 have been described in connection with FIG. 1. As shown in FIG. 2, first machine 110 may include a wireless communication component 210, a collision avoidance device 220, and a user interface 230. Wireless communication component 210 may include one or more devices that are capable of communicating with back office system 120, second machine 130, and/or third machine 140, as described herein.

As an example, wireless communication component 210 may provide the soil property information to back office system 120, second machine 130, and/or third machine 140. Additionally, or alternatively, wireless communication component 210 may provide one or more instructions to second machine 130 and/or third machine 140 to cause second machine 130 and/or third machine 140 to perform one or more actions with respect to the soil (e.g., to adjust the property of the soil). Wireless communication component 210 may include a transceiver, a separate transmitter and receiver, and/or an antenna, among other examples.

Wireless communication component 210 may communicate with back office system 120, second machine 130, and/or third machine 140 using a short-range wireless communication protocol such as, for example, BLUETOOTH® Low-Energy, BLUETOOTH®, Wi-Fi, near-field communication (NFC), Z-Wave, ZigBee, or Institute of Electrical and Electronics Engineers (IEEE) 802.154, among other examples. Additionally, or alternatively, wireless communication component 210 may communicate with back office system 120, second machine 130, and/or third machine 140 via a network that includes one or more wired and/or wireless networks.

Collision avoidance device 220 may include one or more devices that are capable of receiving, generating, storing, processing, and/or providing information to enable first machine 110 to navigate around obstacles located at the job site, to detect when equipment or on-site personnel are approaching first machine 110, to suspend movement or change direction to avoid collision with obstacles, and/or communicate with other machines equipped with a similar device to enable machine-to-machine communication of positions of first machine 110 and positions of the other machines.

User interface 230 may be configured to enable users (e.g., a site operator of a job site) to provide instructions to cause first machine 110 to measure the property of the soil. User device 240 may include one or more devices capable of receiving, generating, storing, processing, and/or providing information associated with measuring the property of the soil, as described elsewhere herein. In some examples, user device 240 may be a device of the site operator. User device 240 may include a communication device and a computing device. User device 240 may include a wireless communication device, a mobile phone, a user equipment, a laptop computer, a tablet computer, a desktop computer, among other examples.

In some implementations, first machine 110 (e.g., controller 114) may obtain measurement information for measuring the property of the soil of the job site. In some examples, first machine 110 may obtain the measurement information from back office system 120. Additionally, or alternatively, first machine 110 may obtain the measurement information via user interface 230. Additionally, or alternatively, first machine 110 may obtain the measurement information from user device 240.

In some implementations, first machine 110 may obtain the measurement information periodically (e.g., every hour, every two hours, every six hours, among other examples). Additionally, or alternatively, first machine 110 may obtain the measurement information based on a trigger (e.g., based on a request from user device 240).

The measurement information may include measurement type information, measurement location information, frequency information, reporting information, and/or target property information, among other examples. The measurement type information may identify a type of measurement to be obtained by first machine 110. For example, first machine 110 may measure the level of moisture of the soil based on the measurement type information identifying a first type of measurement. Alternatively, first machine 110 may measure the density of the soil based on the measurement type information identifying a second type of measurement. Other types of measurements are also contemplated. For example, first machine 110 may measure mechanical properties of the soil (e.g., elastic modulus), a measure of gradation, a temperature, a grade of the job site, and/or a pH level, among other examples.

The measurement location information may identify the location of the soil and/or identify a portion of the job site that includes the soil. The frequency information may identify a frequency of obtaining measurements of the soil (e.g., a frequency of measuring the property of the soil). For example, the frequency information may indicate that first machine 110 is to measure the property of the soil every ten minutes, every thirty minutes, and/or every hour, among other examples.

The reporting information may indicate that the soil property information (regarding the property of the soil) is to be reported. For example, the reporting information may indicate that the soil property information is to be reported to back office system 120, to second machine 130, to third machine 140, and/or to user device 240, among other examples. The target property information may indicate a target property of the soil. The target property of the soil may include a target level of moisture of the soil and/or a target density of the soil.

Based on obtaining the measurement information, first machine 110 may measure the property of the soil. First machine 110 may measure the property of the soil (e.g., using measuring unit 116) based on the measurement information, as explained above. For example, first machine 110 may navigate to the location of the soil or to the portion of the job site that includes the soil and may measure the property of the soil in accordance with the frequency identified by the frequency information.

First machine 110 (e.g., controller 114) may measure the level of moisture of the soil or measure the density of the soil based on the measurement type information. In some instances, the measurement information may indicate that an initial grading operation is being performed at the portion of the job site. In this regard, as part of the initial grading operation, first machine 110 may measure the level of moisture prior to measuring the density of the soil.

In some examples, first machine 110 may generate the soil property information based on measuring the level of moisture. In some instances, based on the reporting information, first machine 110 may provide the soil property information to back office system 120, to second machine 130, to third machine 140, and/or to user device 240, among other examples.

First machine 110 (e.g., controller 114) may compare the property of the soil (measured by first machine 110) and the target property of the soil. For example, first machine 110 may compare the level of moisture and the target level of moisture. For instance, first machine 110 may compare a value, corresponding to the level of moisture of the soil, and one or more value thresholds corresponding to the target level of moisture of the soil. As an example, first machine 110 may compare the value and a first value threshold and/or may compare the value and a second value threshold that is less than the first value threshold.

In some examples, first machine 110 may determine that the level of moisture of the soil exceeds the target level of moisture based on the value satisfying the first value threshold. Alternatively, first machine 110 may determine that the level of moisture of the soil is less than the target level of moisture based on the value not satisfying the second value threshold. Alternatively, first machine 110 may determine that the level of moisture corresponds to the target level of moisture based on the value satisfying the second value threshold but not satisfying the first value threshold. For example, the value may be included in a range of values between the first value threshold and the second value threshold. For instance, first machine 110 may determine that the level of moisture corresponds to the target level of moisture based on the value being equal to a lowest value of the range of values or being equal to a highest value of the range of values.

In some situations, first machine 110 (e.g., controller 114) may generate one or more instructions that cause one or more machines to perform one or more actions with respect to the soil based on comparing the level of moisture of the soil and the target level of moisture. The one or more instructions may include the soil property information. For example, first machine 110 may generate a first instruction to cause a machine to perform a first action to decrease the level of moisture of the soil based on determining that the level of moisture of the soil exceeds the target level of moisture. For instance, the first instruction may cause the machine to break up the soil to enable the moisture to evaporate. In some examples, the machine may be a dozer and the first instruction may cause the dozer to perform a ripping operation, using a ripper of the dozer, to break up the soil to enable the soil to dry. Additionally, or alternatively, to generating the first instruction, first machine 110 may suspend any operation of first machine 110 to enable the soil to dry.

First machine 110 may generate a second instruction to cause another machine to perform a second action to increase the level of moisture of the soil based on determining that the level of moisture of the soil is less than the target level of moisture. For instance, the second instruction may cause second machine 130 to navigate to the location of the soil and deliver water to the location of the soil.

First machine 110 may generate a third instruction to cause a machine to perform a third action to compact the soil based on determining that the level of moisture of the soil corresponds to the target level of moisture. For instance, the third instruction may cause third machine 140 to navigate to the location of the soil and to compact the soil. In some instances, first machine 110 may generate an additional instruction to cause a paving machine to perform a paving operation on the soil. For example, first machine 110 may generate a fourth instruction to cause the paving machine to provide material on the soil as part of the paving operation (e.g., after the soil has been compacted by third machine 140).

In some situations, first machine 110 may determine an evaporation rate of a moisture of the soil. For example, first machine 110 may determine the evaporation rate based on weather information regarding weather conditions at the location of the soil. The weather conditions may include a temperature, a level of humidity, a level of solar irradiance, among other examples. The weather information may be received from second machine 130, third machine 140, back office system 120, and/or user device 240. In some situations, first machine 110 may receive information regarding the evaporation rate from second machine 130, third machine 140, back office system 120, and/or user device 240.

First machine 110 may generate the one or more instructions based on the level of moisture of the soil and the evaporation rate of the soil. For example, first machine 110 may determine that, while the level of moisture corresponds to the target level of moisture, the evaporation rate indicates that the level of moisture will decrease below the target level of moisture within a time threshold. Accordingly, first machine 110 may generate the second instruction.

First machine 110 (e.g., controller 114) may provide the one or more instructions to the one or more machines to cause the one or more machines to perform the one or more actions. For example, first machine 110 may provide the first instruction to the dozer to cause the dozer to perform the ripping operation, may provide the second instruction to second machine 130 to cause second machine 130 to deliver water to the location of the soil, and so on. In some implementations, first machine 110 may provide the soil property information to back office system 120 to cause back office system 120 to generate the instructions and provide the instructions to the one or more machines.

In some implementations, first machine 110 may measure the density of the soil and determine whether the density of the soil corresponds to the target density of the soil prior to a compaction operation being performed on the soil (e.g., prior to generating the third instructions). In some examples, first machine 110 may measure the density of the soil based on determining that the level of moisture of the soil corresponds to the target level of moisture of the soil. Additionally, or alternatively, first machine 110 may measure the density of the soil based on the measurement type information identifying the second type of measurement, as explained above.

Additionally, or alternatively, first machine 110 may measure the density of the soil based on an amount of traffic associated with machines traveling over the soil. For example, first machine 110 may determine the amount of traffic associated with machines traveling over the soil. For instance, first machine 110 may obtain traffic information indicating the amount of traffic from back office system 120, second machine 130, third machine 140, and/or user device 240. Based on the traffic information, first machine 110 may determine the amount of traffic and determine, based on the amount of traffic, that the soil is being compacted. For example, first machine 110 may determine that the soil is being compacted based on the amount of traffic satisfying an amount threshold. First machine 110 may measure the density of the soil based on determining that the soil is being compacted.

First machine 110 may compare the density and the target density, in a manner similar to the manner described above in connection with the target level of moisture. For instance, first machine 110 may compare a value, corresponding to the density, and one or more value thresholds corresponding to the target density of the soil. As an example, first machine 110 may compare the value and a third value threshold and/or may compare the value and a fourth value threshold that is less than the third value threshold.

In some examples, first machine 110 may determine that the density of the soil exceeds the target density based on the value satisfying the third value threshold. Alternatively, first machine 110 may determine that the density of the soil is less than the target density based on the value not satisfying the fourth value threshold. Alternatively, first machine 110 may determine that the density corresponds to the target density based on the value satisfying the fourth value threshold but not satisfying the third value threshold. For example, the value may be included in a range of values between the third value threshold and the fourth value threshold. For instance, the value may be equal to a lowest value of the range of values or may be equal to a highest value of the range of values.

In some situations, first machine 110 may generate one or more instructions that cause one or more machines to perform one or more actions with respect to the soil based on comparing the density and the target density. The one or more instructions may include the soil property information. For example, based on determining that the density is less than the target density, first machine 110 may generate the third instruction to cause third machine 140 to navigate to the location of the soil and to compact the soil.

Alternatively, based on determining that the density corresponds to the target density, first machine 110 may generate the fourth instruction to cause the paving machine to provide material on the soil as part of the paving operation. In some implementations, the third instruction may enable third machine 140 to suspend a compacting operation in a particular portion of the job site that has achieved the target density and focus on another particular portion of the job site that has not achieved the target density. In some implementations, first machine 110 may provide the soil density information to back office system 120 to cause back office system 120 to generate the instructions and provide the instructions to the one or more machines.

In some implementations, first machine 110 may continue to measure the density of the soil until the density corresponds to the target density. First machine 110 may wait at the location of the soil until first machine 110 receives new measuring information for a different portion of the job site. The new measuring information may cause first machine 110 to be redeployed to the other portion of the job site to measure the property of a soil at the other portion of the job site.

The number and arrangement of devices shown in FIG. 2 are provided as an example. In practice, there may be additional devices, fewer devices, different devices, or differently arranged devices than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of the example component may perform one or more functions described as being performed by another set of devices of the example component.

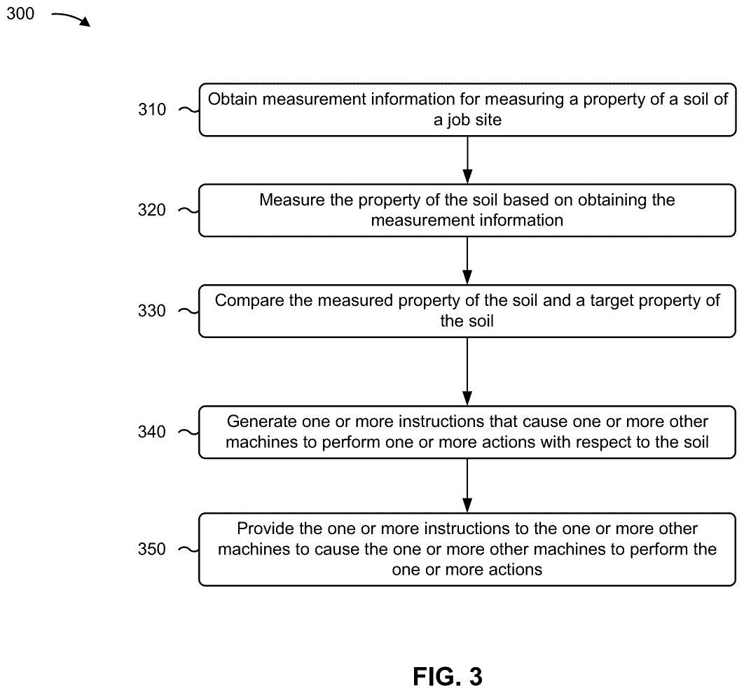

FIG. 3 is a flowchart of an example process 300 relating to determining a property of a soil using a machine. In some implementations, one or more process blocks of FIG. 3 may be performed by a controller (e.g., controller 114). In some implementations, one or more process blocks of FIG. 3 may be performed by another device or a group of devices separate from or including the controller, such as a back office system (e.g., back office system 120), a second machine (e.g., second machine 130), and/or a third machine (e.g., third machine 140).

As shown in FIG. 3, process 300 may include obtaining measurement information for measuring a property of a soil of a job site, wherein the property of the soil includes one or more of a level of moisture of the soil or a density of the soil (block 310). For example, the controller may obtain measurement information for measuring a property of a soil of a job site, wherein the property of the soil includes one or more of a level of moisture of the soil or a density of the soil, as described above. In some implementations, the property of the soil includes one or more of a level of moisture of the soil or a density of the soil.

The measurement information includes measurement type information identifying a type of measurement to be obtained by the machine. Additionally, or alternatively, the measurement information includes frequency information identifying a frequency of measuring the level of moisture.

As further shown in FIG. 3, process 300 may include measuring the property of the soil based on obtaining the measurement information (block 320). For example, the controller may measure the property of the soil based on obtaining the measurement information, as described above.

Measuring the property of the soil comprises measuring the property of the soil in accordance with the frequency identified by the frequency information. Measuring the property of the soil comprises measuring the level of moisture of the soil when the measurement type information identifies a first type of measurement, and measuring the density of the soil when the measurement type information identifies a second type of measurement.

As further shown in FIG. 3, process 300 may include comparing the measured property of the soil and a target property of the soil, wherein the target property of the soil includes one or more of a target level of moisture of the soil or a target density of the soil (block 330). For example, the controller may compare the measured property of the soil and a target property of the soil, wherein the target property of the soil includes one or more of a target level of moisture of the soil or a target density of the soil, as described above. In some implementations, the target property of the soil includes one or more of a target level of moisture of the soil or a target density of the soil.

As further shown in FIG. 3, process 300 may include generating one or more instructions that cause one or more other machines to perform one or more actions with respect to the soil, wherein the one or more instructions are generated based on comparing the measured property of the soil and the target property (block 340). For example, the controller may generate one or more instructions that cause one or more other machines to perform one or more actions with respect to the soil, wherein the one or more instructions are generated based on comparing the measured property of the soil and the target property, as described above. In some implementations, the one or more instructions are generated based on comparing the measured property of the soil and the target property.

In some examples, the machine is a first machine. Comparing the property of the soil and the target property of the soil comprises comparing a value, corresponding to the property of the soil, and one or more value thresholds corresponding to the target property of the soil. Generating the one or more instructions comprises generating a first instruction to cause a second machine to perform a first action to decrease the level of moisture of the soil based on the value satisfying a first value threshold of the one or more value thresholds, or generating a second instruction to cause a third machine to perform a second action to increase the level of moisture of the soil based on the value not satisfying a second value threshold, of the one or more value thresholds, that is less than the first value threshold.

Generating the one or more instructions further comprises generating a third instruction to cause a fourth machine to perform a third action to compact the soil based on the value not satisfying the first value threshold and satisfying the second value threshold.

As further shown in FIG. 3, process 300 may include providing the one or more instructions to the one or more other machines to cause the one or more other machines to perform the one or more actions (block 350). For example, the controller may provide the one or more instructions to the one or more other machines to cause the one or more other machines to perform the one or more actions, as described above.

Providing the one or more instructions comprises providing the first instruction to the second machine, or providing the second instruction to the third machine, wherein the third machine is different than the second machine. Providing the one or more instructions comprises providing the third instruction to the fourth machine.

Process 300 includes providing soil property information to one or more devices. The soil property information includes information identifying the property of the soil and information identifying a location of the soil.

In some implementations, process 300 includes determining an amount of traffic associated with machines traveling over the soil, determining, based on the amount of traffic, that the soil is being compacted, and determining the density of the soil based on determining that the soil is being compacted.

Although FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel.

### INDUSTRIAL APPLICABILITY

Implementations described herein are directed to using a machine to measure a property of a soil of a job site. For example, the machine may measure a level of moisture of the soil and/or a density of the soil.

Typically, measuring the level of moisture and measuring the level of density are performed manually using a measuring device. The manual process may be performed for only a small section of the job site (e.g., 1% of the job site). The manual process is a time-consuming process. Accordingly, the manual process consumes computing resources associated with obtaining measurements manually, storage resources associated with storing the measurements, and/or network resources, among other examples.

Additionally, the manual process is subject to errors. Accordingly, the manual process results in using one or more machines to correct compaction issues. Additionally, correcting the compaction issue is a costly process. For example, repairing a soil base (after a structure has been built on the soil bases) is a time-consuming and costly process. As explained above, the later the discovery of the compaction issue occurs, the more time consuming and costly the correction process becomes.

Using the machine to measure the level of moisture or the level of density enables measurements to be obtained for sections of the job site. A size of each section of the job typically exceeds a size of a section of a job site for which the property of a soil is measured using the manual process. Therefore, using the machine to measure the property of the soil preserves computing resources associated with obtaining measurements of the property of the soil manually, storage resources associated with storing the measurements, and/or network resources, among other examples, that would have otherwise been consumed by the manual process.

Using the machine to measure the property of the soil provides additional benefits. For example, using the machine as described herein increases an amount of and a frequency of information obtained (and/or provided) in real time. Such an increase improves a speed at which decisions, at the job site, are made, thereby improving a measure of confidence in the quality of work completed at the job site. Using the machine as described herein may increase an efficiency of the job site based on increased coverage of measurements obtained by the machine. Using the machine as described herein may additionally increase a frequency with which compaction issues are discovered and may allow appropriate responses to correct the compaction issue prior subsequent work being performed at the job site, thereby eliminating waste. Using the machine as described herein may reduce time spent on compaction by determining whether a compaction target has been met, in a compaction process, earlier than expected. Using the machine as described herein may save time, fuel, and wear of compaction machines. Additionally, the compaction machines may be deployed to another portion of the job site and/or to other job sites.

The foregoing disclosure provides illustration and description, but is not intended to be exhaustive or to limit the implementations to the precise forms disclosed. Modifications and variations may be made in light of the above disclosure or may be acquired from practice of the implementations. Furthermore, any of the implementations described herein may be combined unless the foregoing disclosure expressly provides a reason that one or more implementations cannot be combined. Even though particular combinations of features are recited in the claims and/or disclosed in the specification, these combinations are not intended to limit the disclosure of various implementations. Although each dependent claim listed below may directly depend on only one claim, the disclosure of various implementations includes each dependent claim in combination with every other claim in the claim set.

As used herein, “a,” “an,” and a “set” are intended to include one or more items, and may be used interchangeably with “one or more.” Further, as used herein, the article “the” is intended to include one or more items referenced in connection with the article “the” and may be used interchangeably with “the one or more.” Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”). Further, spatially relative terms, such as “below,” “lower,” “above,” “upper,” and the like, may be used herein for ease of description to describe one element or feature's relationship to another element(s) or feature(s) as illustrated in the figures. The spatially relative terms are intended to encompass different orientations of the apparatus, device, and/or element in use or operation in addition to the orientation depicted in the figures. The apparatus may be otherwise oriented (rotated 90 degrees or at other orientations) and the spatially relative descriptors used herein may likewise be interpreted accordingly.

What is claimed is:

1. A method performed by a controller of a machine, the method comprising:
obtaining measurement information for measuring a property of a soil of a job site,
wherein the property of the soil includes one or more of a level of moisture of the soil or a density of the soil;
measuring the property of the soil based on obtaining the measurement information;
comparing the measured property of the soil and a target property of the soil,
wherein the target property of the soil includes one or more of a target level of moisture of the soil or a target density of the soil;
generating one or more instructions that cause one or more other machines to perform one or more actions with respect to the soil,
wherein the one or more instructions are generated based on comparing the measured property of the soil and the target property; and
providing the one or more instructions to the one or more other machines to cause the one or more other machines to perform the one or more actions.

2. The method of claim 1, wherein the machine is a first machine,
wherein comparing the property of the soil and the target property of the soil comprises:
comparing a value, corresponding to the property of the soil, and one or more value thresholds corresponding to the target property of the soil, and
wherein generating the one or more instructions comprises:
generating a first instruction to cause a second machine to perform a first action to decrease the level of moisture of the soil based on the value satisfying a first value threshold of the one or more value thresholds; or
generating a second instruction to cause a third machine to perform a second action to increase the level of moisture of the soil based on the value not satisfying a second value threshold, of the one or more value thresholds, that is less than the first value threshold, and
wherein providing the one or more instructions comprises:
providing the first instruction to the second machine; or
providing the second instruction to the third machine,
wherein the third machine is different than the second machine.

3. The method of claim 2, wherein generating the one or more instructions further comprises:
generating a third instruction to cause a fourth machine to perform a third action to compact the soil based on the value not satisfying the first value threshold and satisfying the second value threshold; and
wherein providing the one or more instructions comprises:
providing the third instruction to the fourth machine.

4. The method of claim 1, further comprising:
providing soil property information to at least one of the one or more other machines or a back office system,
wherein the soil property information includes information identifying the property of the soil and information identifying a location of the soil.

5. The method of claim 1, wherein the measurement information includes frequency information identifying a frequency of measuring one or more of the level of moisture or the density of the soil, and
wherein measuring the property of the soil comprises:
measuring the property of the soil in accordance with the frequency identified by the frequency information.

6. The method of claim 1, wherein the measurement information includes measurement type information identifying a type of measurement to be obtained by the machine, and
wherein measuring the property of the soil comprises:
measuring the level of moisture of the soil when the measurement type information identifies a first type of measurement; and
measuring the density of the soil when the measurement type information identifies a second type of measurement.

7. The method of claim 1, further comprising:
determining an amount of traffic associated with machines traveling over the soil;
determining, based on the amount of traffic, that the soil is being compacted; and
determining the density of the soil based on determining that the soil is being compacted.

8. A controller associated with a first machine, the controller comprising:
one or more memories; and
one or more processors configured to:
obtain measurement information for measuring a property of a soil of a job site,
wherein the property of the soil includes one or more of a level of moisture of the soil or a density of the soil;
measure the property of the soil based on receiving the measurement information;
compare the property of the soil and a target property of the soil,
wherein the target property of the soil includes one or more of a target level of moisture of the soil or a target density of the soil;
generate an instruction that causes a second machine to perform an action with respect to the soil to adjust the property of the soil with respect to the target property,
wherein the instruction is generated based on comparing the property of the soil and the target property; and
provide the instruction to the second machine to cause the second machine to perform the action to adjust the property of the soil.

9. The controller of claim 8, wherein the one or more processors are further configured to:
measure the property of the soil an additional time after providing the instruction;
determine that the property of the soil is different than the target property of the soil based on measuring the property of the soil an additional time; and
measure the property of the soil one or more additional times based on determining that the property of the soil is different than the target property of the soil.

10. The controller of claim 8, wherein, to measure the property of the soil, the one or more processors are further configured to:
determine that the level of moisture of the soil corresponds to the target level of moisture; and
measure the density of the soil based on determining that the level of moisture of the soil corresponds to the target level of moisture.

11. The controller of claim 8, wherein the measurement information includes reporting information indicating that soil property information regarding the property of the soil is to be reported,
wherein the soil property information includes information indicating the property of the soil and information identifying a location of the soil, and
wherein, based on the reporting information, the one or more processors are further configured to at least one of:
provide soil property information to one or more devices that monitor operations at the job site; or
provide the instruction to the second machine,
wherein the instruction includes the soil property information.

12. The controller of claim 8, wherein, to generate the instruction, the one or more processors are further configured to:
determine the level of moisture of the soil;
determine an evaporation rate of a moisture of the soil; and
generate the instruction based on the level of moisture of the soil and the evaporation rate of the soil.

13. The controller of claim 8, wherein, to compare the property of the soil and the target property of the soil, the one or more processors are further configured to:
compare a value, corresponding to the property of the soil, and value thresholds corresponding to the target property of the soil, and
wherein, to generate the instruction, the one or more processors are further configured to:
generate a first instruction to decrease the level of moisture of the soil based on the value satisfying a first value threshold of the value thresholds; and
generate a second instruction to increase the level of moisture of the soil based on the value not satisfying a second value threshold, of the value thresholds, that is less than the first value threshold.

14. The controller of claim 13, wherein, to generate the instruction, the one or more processors are further configured to:
generate a third instruction to compact the soil based on the value not satisfying the first value threshold and satisfying the second value threshold; or
generate a fourth instruction to provide material on the soil based on the value not satisfying the first value threshold and satisfying the second value threshold.

15. A machine comprising:
a plurality of ground engaging members configured to propel the machine at a job site;
a measuring unit configured to measure a property of a soil of the job site; and
a controller configured to:
obtain measurement information for measuring the property of the soil;
measure the property of the soil, using the measuring unit, based on receiving the measurement information;
compare a value of the property of the soil and value thresholds associated with a target property of the soil;
generate an instruction based on comparing the value and the value thresholds,
wherein the instruction is a first instruction based on the value satisfying a first value threshold of the value thresholds,
wherein the instruction is a second instruction based on the value not satisfying a second value threshold, of the value thresholds, that is less than the first value threshold, or
wherein the instruction is a third instruction based on the value not satisfying the first value threshold and satisfying the second value threshold; and
provide the instruction to one or more machines.

16. The machine of claim 15, wherein, to provide the instruction, the controller is configured to:
provide the instruction to a machine of a first type based on the instruction being the first instruction;
provide the instruction to a machine of a second type different than the first type based on the instruction being the second instruction; or
provide the instruction to a machine of a third type based on the instruction being the third instruction.

17. The machine of claim 15, wherein, to provide the instruction, the controller is configured to:
determine an amount of traffic associated with machines traveling over the soil;
determine, based on the amount of traffic, that the soil is being compacted; and
determine a density of the soil based on determining that the soil is being compacted.

18. The machine of claim 15, wherein the controller is configured to:
provide soil property information to one or more devices,
wherein the soil property information includes information identifying the property of the soil and information identifying a location of the machine.

19. The machine of claim 15, wherein, to measure the property of the soil, the controller is further configured to:
determine that a level of moisture of the soil corresponds to a target level of moisture; and
measure a density of the soil based on determining that the level of moisture of the soil corresponds to the target level of moisture.

20. The machine of claim 15, wherein the measurement information includes measurement type information identifying a type of measurement to be obtained, and
wherein, to measure the property of the soil, the controller is configured to:
measure a level of moisture of the soil based on the measurement type information identifying a first type of measurement; and
measure a density of the soil based on the measurement type information identifying a second type of measurement.
